(19) United States
(12) Patent Application Publication
(10) Pub. No.: US 2024/0318121 A1
(43) Pub. Date: Sep. 26, 2024

(54) METHOD AND APPARATUS FOR STERILE AND NONINVASIVE MEASUREMENTS OF SUBSTANCES IN BIOREACTORS AND OTHER STERILE ENVIRONMENTS

(71) Applicant: UNIVERSITY OF MARYLAND, BALTIMORE COUNTY, Baltimore, MD (US)

(72) Inventors: Govind RAO, Ellicott City, MD (US); Vida Rahmatnejad, Baltimore, MD (US); Xudong Ge, Woodstock, MD (US); Vikash Kumar, Castonsville, MD (US); Michael Tolosa, Columbia, MD (US)

(21) Appl. No.: 18/668,438

(22) Filed: May 20, 2024

Related U.S. Application Data
(63) Continuation of application No. PCT/US2022/080899, filed on Dec. 5, 2022.
(60) Provisional application No. 63/286,219, filed on Dec. 6, 2021.
(60) Provisional application No. 63/287,138, filed on Dec. 8, 2021.
(60) Provisional application No. 63/347,739, filed on Jun. 1, 2022.

(51) Int. Cl. C12M 1/34; C12M 1/00

(52) U.S. Cl. CPC C12M 41/34; C12M 29/04; C12M 41/26

(57) ABSTRACT

The present invention provides for systems and method for noninvasive measurement and monitoring of cell culture parameters including dissolved oxygen (DO), dissolved carbon dioxide (DCO₂), and pH wherein there is no direct contact with the cell culture environment which is achieved by conducting the measurements and monitoring on the outside of the bioreactor through semi-permeable membranes incorporated into the system to enhance the process and eliminate the risk of contamination associated with invasive sensors.

### CROSS REFERENCE TO RELATED APPLICATION

This application is filed under the provisions of 35 U.S.C. § 120 and is a continuation-in-part of International Patent Application No. PCT/US2022/080899, filed on 5 Dec. 2022 entitled “METHOD AND APPARATUS FOR STERILE AND NONINVASIVE MEASUREMENTS OF SUBSTANCES IN BIOREACTORS AND OTHER STERILE ENVIRONMENTS” in the name of Govind RAO, et al., which claims priority to U.S. Provisional Patent Application No. 63/286,219 filed on Dec. 6, 2021, U.S. Provisional Patent Application No. 63/287,138 filed Dec. 8, 2021, and U.S. Provisional Patent Application No. 63/347,739 filed Jun. 1, 2022, the contents of which are hereby incorporated by reference herein for all purposes.

### TECHNICAL FIELD

The present invention provides for systems and method for noninvasive measurement and monitoring of cell culture parameters including dissolved oxygen (DO), dissolved carbon dioxide (DCO₂), and pH wherein there is no direct contact with the cell culture environment within a bioprocess bioreactor or contain and is achieved by conducting the measurements and monitoring through semi-permeable membranes incorporated on the outside of the bioprocess bioreactor container to enhance the process and eliminate the risk of contamination associated with invasive sensors.

### BACKGROUND OF THE INVENTION

### Related Art

Bioreactors and other similar sterile environments require many critical process parameters such as glucose, oxygen, pH, CO₂, and other small and large molecules to be continuously measured. Further, bioreactors are valuable platforms for cost-effective and consistent production of cell therapies as they maintain the culture environment [1][3]. Cell therapy is a therapy where cellular materials are injected, grafted or implanted into the body of the patient in order to effectuate medicinal effect. This method is increasingly becoming a part of the medical practice and has applications in various diseases ranging from diabetes and wounds of soft tissues to nervous system, genetic disorders, and cancer [9]. Despite the promising effect that cell therapies have, they are associated with significant issues such as having poorly defined manufacturing processes, lack of effective small-scale models, and high costs [2][7]. Manufacturing cells for cell therapies is a delicate process and is associated with modifications to the cells at specific time points. Cell culture is the longest step throughout the manufacturing process, and cell characteristics could be affected during this step. The fact that cell quality is a critical factor defining the therapeutic efficacy of cell therapies makes cell culture one of the most critical steps in the manufacturing process of cell therapies.

In addition, monitoring cell culture processes is an essential factor in achieving the optimal performance and consistency. Monitoring normally happens by placing sensors inside the cell culture environment and tracking the changes in different parameters such as temperature, pH, and dissolved oxygen (DO) [25]. Application of sensors helps in improving cell expansion, optimizing the product, enhancing the process yield, identifying the problems, and mitigating them at early stages. Additionally, monitoring systems are helpful in simplifying the process validation, and improving the reproducibility of the production [9][4]. However, the presence of sensors placed in cell culture environment could result in contamination [25]. Additionally, these sensors are comprised of polymers with immobilized dyes, metal ions etc. and post a risk of molecules leaching and/or being extracted into the process fluid. Contamination in cell therapy products compromises the product quality and causes immunogenic risks in patient. Leachables and extractables from sensors being transferred into the patient is also undesirable. Given the fact that these products cannot be terminally sterilized because of their large size and being fragile, eliminating the risk of cross-contamination during the cell culture process is more critical compared to other biologics [3]. Studies show that open processes to the environment is the main reason for contamination [5].

Notably, easily implemented approaches to overcome the actual contamination threat is currently unavailable. As such, there is a need for a simple, robust method and system to monitoring cell culture parameters in a noninvasive way and the present invention provides such methods and systems to overcome contamination of products. It confers the additional advantage of minimizing leachables and extractables entering into the product.

### SUMMARY OF THE INVENTION

The present invention provides for a noninvasive systems and methods to measure and monitor detectable parameters within a bioprocess medium such as a cell culture without contaminating the cell culture and without direct contact with cell culture and components therein. All testing is conducted outside of a bioprocess container in light of the fact that testing detectable parameters diffuse or flow through a semipermeable membrane positioned on the outside of the bioprocess container.

In one aspect, the present invention provides for a noninvasive system for monitoring and/or measuring testing parameters within a bioprocess medium, the noninvasive system comprising:
a bioprocess container for holding the bioprocess medium, wherein the bioprocess container comprises an (i) opening in a wall of the bioprocess container for monitoring and/or measuring testing parameters diffusing from the bioprocess container, (ii) a waste line attached to the bioprocess container for movement of fluid comprising the testing parameters from the bioprocess container or (iii) a recirculation loop for movement of fluid comprising the testing parameters to and from the bioprocess container;
a semi-permeable membrane communicatively connected to a sampler receptor wherein the semipermeable membrane is positioned between the waste line, opening or recirculation loop and the sampler receptor and allows for movement of testing parameters into the sampler receptor; and
a sensor communicatively connected to the sampler receptor for measuring and/or monitoring the testing parameters within the bioprocess medium.

The above system provides for a closed bioprocess system with all testing is conducted and relevant sensors are placed outside of the bioprocess container. Importantly there are no sensors or testing aspect located within the bioprocess container. Further, the semi-permeable membranes are on the outside of the bioprocess container, waste line or recirculation loop. Regarding an opening in the bioprocess container, it is preferably positioned at the bottom of the bioprocess container or certainly in the lower wall areas of the container. It is understood that this opening is not the same as an inlet for introducing a bioprocess medium into the bioprocess container. Preferably the bioprocess container and lines extending therefrom are fabricated with nonpermeable materials, so that testing of parameters is only available after testing parameters passes through the semipermeable membrane. Preferably the waste line, opening or recirculation loop includes flow controls to control the flow of fluids and provide the option of testing when necessary.

In the above system, the semipermeable membrane is fabricated of silicone, cellulose and other materials that are permeable to DCO₂, DO and pH.

In another aspect, the present invention provides for a noninvasive system for monitoring and/or measuring testing parameters including but not limited to dissolved O₂, pH and dissolved CO₂ within a bioprocess medium, the noninvasive system comprising:
a bioprocess container for holding the bioprocess medium, wherein the bioprocess container is connected to a waste line or recirculation loop line for movement of fluid from the bioprocess container;
at least one sampling chamber for each testing parameter, wherein the sampling chamber has an inlet and outlet and communicatively connected to the waste line or recirculation loop, wherein the outlet is communicatively connected to a sampler receptor;
a semi-permeable membrane positioned between the outlet of the sampling chamber and the sampler receptor and allows for movement of fluid from the sampling chamber into the one sampler receptor; and
a sensor communicatively connected to the at least one sampler receptor for measuring and/or monitoring the testing parameters of at least dissolved O₂, pH and dissolved CO₂ within the bioprocess medium.

The sampler receptors collect the medium for testing of the components of DO, DCO₂, and pH, after the bioprocess medium passes through the semi-permeable membranes.

During the measurement process, the medium is transferred to flow/waste line attached to individual chambers, the number relative to the at least three testing procedures. Then a specific volume of medium is collected in the individual chambers. A membrane is attached to the bottom of each chamber. Thus, CO₂, O₂ or protons will diffuse through their corresponding membranes. After this step, the measurements are conducted via DO, DCO₂, and pH sensors. The semipermeable membranes for DO, and DCO₂ is preferably silicone and for pH is a cellulose membrane.

Preferably the system above includes three sampling chambers each having one inlet and one outlet, wherein the outlet is connected to a membrane specific for measuring the components of DO, DCO₂, and pH, wherein the first chamber is covered with cellulose membrane, and is allocated for measuring pH, wherein the outlet of the second chamber is covered with silicone membrane which is highly permeable to oxygen to measure and the outlet of the third chamber is similarly covered with silicone membrane via which DCO₂ is measured. The chambers are fabricated of a nonpermeable material. Each membrane is communicatively connected to a sensor specific for measuring the investigated components. The culture medium is moved into a flow channel for movement into each chamber, wherein each of the chambers are connected to each other through the flow line and each is provided with an effective testing amount of the medium.

For the pH sensor, a pH patch is communicatively connected to cellulose membrane. The pH patch preferably comprises a fluorescent dye that is immobilized in an anion exchange resin. The resin is then entrapped into a hydrogel highly permeable to protons. After electromagnetic activation, the pH value is relative to the fluorescent signal. For measuring the DO and DCO₂ components the sensor is preferably an optical sensor including electronics and a sensing patch comprising a fluorescent dye immobilized in a silicone matrix.

Another aspect of the present invention provides for a method for noninvasive monitoring and/or measuring testing parameters including but not limited DCO₂, DO and pH within a bioprocess medium, the method comprising:
i) providing a noninvasive system comprising:
   a) a bioprocess container for holding the bioprocess medium, wherein the bioprocess container is connected to a waste line or recirculation loop line for movement of fluid from the bioprocess container;
   b) at least one sampling chamber for each testing parameter, wherein the sampling chamber has an inlet and outlet and communicatively connected to the waste line or recirculation loop, wherein the outlet is communicatively connected to a sampler receptor;
   c) a semi-permeable membrane positioned between the outlet of the sampling chamber and the sampler receptor and allows for movement of fluid from the sampling chamber into the one sampler receptor; and
   d) a sensor communicatively connected to the at least one sampler receptor for measuring and/or monitoring the testing parameters of at least dissolved O₂, pH and dissolved CO₂ within the bioprocess medium;
ii) moving/flowing the bioprocess medium from the bioprocess container into the at least one sampling chamber, wherein the bioprocess medium passes through the outlet of the sampling chamber and through the semi-permeable membrane, wherein the semipermeable membrane is fabricated of silicone, cellulose and other materials that are permeable to DCO₂, DO and pH, respectively;
iii) measuring the testing parameter in the sampler receptor with a testing sensor.

In yet another aspect, the present invention provides for a non-invasive system for monitoring and/or measuring testing parameters including but not limited DCO₂, DO and pH within a bioprocess medium, the method comprising:
a bioprocess container for holding the bioprocess medium, wherein the bioprocess container has an inlet for moving in bioprocess medium and an outlet;
a semi-permeable membrane positioned between the outlet of the bioprocess container and a sampler receptor and allows for diffusion of testing parameters from the bioprocess medium into the sampler receptor; and
a sensor communicatively connected to the sampler receptor for measuring and/or monitoring the testing parameters of at least dissolved O₂, pH and dissolved CO₂ within the bioprocess medium. Again, all testing and sensors is conducted outside of the bioprocess container thereby eliminating any possible contamination of the bioprocess medium during the testing.

Other features and advantages of the invention will be apparent from the following detailed description, drawings, and claims.

### BRIEF DESCRIPTION OF THE FIGURES

FIG. 1A shows a hole is created in the bottom wall of a T-flask. A silicone membrane and a sampler are then attached on the hole from outside.

FIG. 1B shows a sideview of the T-flask where CO₂ present in the cell culture medium passes through the hole, and silicone membrane. It is then collected in the sampler and transferred to the sensor.

FIG. 1C shows a photo showing a modified T-flask where impermeable tubes are attached to the sampler for noninvasive measurement of the CO₂ from cell culture.

FIG. 2A shows dissolved oxygen and dissolved CO₂ profiles for Pichia pastoris culture. Dissolved CO₂ profile obtained using the noninvasive method.

FIG. 2B shows dissolved oxygen, pH, and dissolved CO₂ for CHO culture. Dissolved CO₂ profile obtained from noninvasive method.

FIG. 3 shows a schematic diagram of the optical pH sensor.

FIG. 4 shows pH patched attached inside a shake flask. The shake flask is then placed on a coaster for measurements.

FIG. 5 shows a method to measure the pH in a noninvasive way, a cellulose membrane was attached on a hole created in the bottom wall of the T-flask. A wet pH patch was then attached on a transparent surface. The surface with pH patch on it, was attached to the cellulose membrane covering the hole from outside. This was conducted in a way that the wet pH patch would be in direct contact with the cellulose membrane covering the hole.

FIG. 6 shows corrected ratios obtained from control method (blue) versus corrected ratios obtained via noninvasive method (cyan). The control method refers to condition when the pH patch is in direct contact with the solution. The noninvasive method refers to the condition where measurements are conducted via cellulose membrane.

FIG. 7 shows the corrected ratio profiles via control method (blue), and via noninvasive method (cyan). The control method refers to condition when the pH patch is in direct contact with the cell culture medium. The noninvasive method refers to the condition where measurements are conducted via cellulose membrane.

FIG. 8 shows a DO patch placed inside a sealing material and then attached to the vessel from outside.

FIG. 9 shows a schematic of the inventive design for noninvasive measurement of DCO₂, DO and pH throughout the cell culture process. Different parts with order of assembly in the flow cell are labeled.

FIG. 10A shows a simplified system for testing of bioprocess parameters in a waste stream.

FIG. 10B shows a simplified system for testing of bioprocess parameters in a recirculating flow system wherein the testing parameters including testing of DCO₂, DO and/or pH.

### DETAILED DESCRIPTION OF THE INVENTION

Various embodiments of the disclosure will be described in detail with reference to figures. Reference to various embodiments does not limit the scope of the invention. Additionally, any examples set forth in this specification are not limiting and merely set forth some of the many possible embodiments of the claimed invention.

All scientific and technical terms used herein have meanings commonly used in the art unless otherwise specified. The definitions provided herein are to facilitate understanding of certain terms used frequently herein and are not meant to limit the scope of the present disclosure.

“Opening” as used herein encompasses, but not limited to a hole, vent, slit, slot, and aperture.

“Noninvasive” as used herein means the monitoring and measuring of different parameters without direct contact with the cell culture environment contained within the bioprocess container or bioreactor.

“Include,” “includes,” or like terms means encompassing but not limited to, that is, inclusive and not exclusive.

“Optional” or “optionally” means that the subsequently described step, feature, condition, characteristic, or structure, occurs/is present or does not occur/is not present, while still being within the scope described.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “has”, “having”, “include”, “includes”, “including”, “comprise”, “comprises”, “comprising” or the like are used in their open-ended inclusive sense, and generally mean “include, but not limited to”, “includes, but not limited to”, or “including, but not limited to”.

While various features, elements or steps of particular embodiments may be disclosed using the transitional phrase “comprising,” it is to be understood that alternative embodiments, including those that may be described using the transitional phrases “consisting of” or “consisting essentially of” are implied.

The present invention provides for a system capable of monitoring cell culture parameters in a noninvasive way to enhance the process and eliminate the risk of contamination associated with invasive sensors. The present monitoring system becomes specifically helpful in circumstances that malfunction is observed in the sensor. In such condition, the sensor will be easily replaced eliminating loss of data. This is another advantage of a noninvasive monitoring system. The noninvasive process monitoring of the present invention is beneficial for cell culture processes in cellbag bioreactors. This type of bioreactor is mainly used for culturing shear sensitive cell types such as T cells. To minimize the shear stress, a rocking motion is used to gently mix the cell culture medium. The rocking motion applied to the cellbag results in the movement of the liquid covering sensors attached to the bottom of the bag. Clearly, the changing location of the liquid leads to more monitoring challenges compared to stirred tank bioreactor. However, the present invention overcomes these issues because monitoring occurs outside of the cellbag.

Cell therapy is a therapy where cellular materials are injected, grafted or implanted into the body of the patient in order to effectuate medicinal effect. This method is increasingly becoming a part of the medical practice and has applications in various diseases ranging from diabetes and wounds of soft tissues to nervous system, genetic disorders, and cancer. Despite the promising effect that cell therapies have, they are associated with significant issues such as having poorly defined manufacturing processes, lack of effective small-scale models, and high costs. Cell culture is the longest step throughout the manufacturing process, and cell characteristics could be affected during this step. The fact that cell quality is a critical factor defining the therapeutic efficacy of cell therapies makes cell culture one of the most critical steps in the manufacturing process of cell therapies.

To address the aforementioned issues, the present invention provides a method for noninvasive measurement of at least dissolved oxygen (DO), dissolved carbon dioxide (DCO₂), and pH. Other parameters for measuring or monitoring include small molecule analytes, proteins and or glucose. As stated above, the term noninvasive refers to “no direct contact with the cell culture environment” and the present invention achieved this advantage by conducting the measurements through semi-permeable membranes.

In one embodiment the membranes are embedded in a uniform flow cell, the flow cell is then integrated with the tube transferring the used medium from bioreactor to the waste. The individual monitoring methods for noninvasive measurement of DO, pH and DCO₂ are fully described below and then a combination for combining three noninvasive sensors at a single release of fluid and simultaneously tested.

### Importance of DO, DCO₂ and pH in Mammalian Cell Culture Processes

In mammalian cultures, metabolism is summarized in two processes: first, respiration where glucose oxidization happens and second, the process that leads to the cell growth. In both of these processes, carbon dioxide (CO₂) is produced as a result of the glucose reacting with oxygen. Some part of the CO₂ produced is consumed in the formation of fatty acids as well as cell membrane; however, the rest is released in the surrounding medium leading to increase in the dissolved CO₂ level in the cell culture environment. An increase in DCO₂ can change intracellular pH, disturb the metabolic activity of cells, decrease the productivity of process, and even result in apoptosis. Therefore, high levels of this parameter can affect cell culture processes by having inhibitory effects on the cell growth and producing therapeutics with low quality and effectiveness [11][12]. For example, some studies show that in T cell culture processes, low levels of CO₂ have negative impact on viability as well as metabolism of cells. As a result, CO₂ is considered a critical process parameter in production process of biopharmaceuticals [23][10].

Oxygen is another important factor in the metabolism of cells. Lack of sufficient oxygen results in alteration in the rate and pattern of the metabolism. On the other hand, excess oxygen in cell culture environment can result in decrease in cell proliferation. Interestingly, some studies report an enhancement in the differentiation of stem cells at low levels of oxygen in the cell culture environment. However, there are studies showing that low levels of oxygen can play role in maintaining the undifferentiated state of stem cells [6][16]. The contradictory findings from the aforementioned studies suggest a threshold-mediated response. In other words, the response of stem cells to different concentrations of oxygen above and beyond a specific limit could be different. Similarly, there is evidence that low levels of oxygen inhibit apoptosis resulted from serum deprivation in mesenchymal stem cells, and high levels of oxygen increase the rate of hematopoietic stem cells exiting quiescence [15][14].

Besides factors such as temperature, pH of the cell microenvironment is another factor affecting enzyme activity. The maximum enzymatic activity of cells is achieved at their optimal pH level, and any deviation from the optimal pH results in a decrease of the enzymatic activity. In addition to dependency on an optimal pH value, each enzyme is functional at a specific range [18]. The pH value of cell culture medium or extracellular pH aligns with intracellular pH. Therefore, the pH value of the cell culture environment will affect enzymes and consequently cellular metabolism. Considering this fact, it is important to maintain the pH of cell culture medium within a physiological range, 6.8-7.4, to maximize the cell viability and productivity. Maintaining pH throughout the stem cell culture process is even more important as it is observed that a 0.5-unit decrease of pH in the microenvironment of human mesenchymal stem cells adversely affects the osteogenic differentiation in osteoprogenitor cells [13].

In light of the above discussion, it can be concluded that levels of DO, DCO₂ and PH are very important in defining the outcome of the cell culture process. Therefore, it is important to monitor the changes in these parameters and control them within a specific range.

### Importance of Noninvasive Monitoring of DO, DCO₂ and pH in Cell Therapy

Despite the aforementioned roles that DO, DCO₂, and pH play in cell behavior, cell culture studies often fail to measure and report these parameters. This results in the lack of detailed and quantitative information from cell culture processes which makes them less repeatable and reliable. Reproducibility and consistency are specifically important for cell therapies when designing a GMP-compliant process as cell therapy facilities are required to manufacture high quality cellular products. This emphasizes on the importance of process analytical techniques (PAT) as they are useful tools for quality control and quality assurance purposes. Model predictive control (MPC) is another useful tool for improving the process by providing mathematical predictions of outcomes. However, MPC techniques are not common in mammalian cell culture processes including cell therapy due to the lack of appropriate monitoring tools [7][9][17]. The aforementioned facts emphasize on the importance of monitoring systems in cell therapy manufacturing processes. Additionally, for each step in cell therapies manufacturing process, the overall approach must include reducing the risk of contamination. Therefore, the closed system of the present invention is certainly more appropriate systems in cell therapies. Considering these points, monitoring DO, DCO₂, and pH in the cell therapy manufacturing process in a noninvasive way addresses the needs and resolving issues associated with the previously used invasive methods.

### Method for Noninvasive Measurement of Dissolved CO₂

The measuring the CO₂ dissolved in the cell culture medium includes the use of a semipermeable membrane that is allows for dissolved CO₂ (DCO₂) in the medium to diffuse through the silicone layer which is collected in a sampling receptor or loop and then measured with a sensor for dissolved CO₂.

A method for measuring the CO₂ dissolved in a cell culture medium includes the discovery that a silicone membrane is permeable to CO₂. Therefore, during the cell culture process, the CO₂ dissolved in the cell culture medium diffuses through the silicone membrane and collected in a sampler for measurement by a sensor. The mass balance equation for the system including the silicone membrane, volumes inside the lines and sensors and tubes is written as:

$$V \frac{dC}{dt} = KA ( {C}_{g} - C ) \qquad ( 1 )$$

Where V is the total volume of the system, C is the CO₂ concentration in the sampling loop, t is time, k is the mass transfer coefficient, A is the total mass transfer area or the area of the silicone layer that is in contact with cell culture medium, and Cg is the CO₂ concentration in the culture medium. Considering that the CO₂ concentration in the sampling loop is zero in the beginning of the recirculation step, the relation below can be concluded:

$${{{C}_{g} = \frac{V}{kA} \frac{dC}{dt} \vert}_{t = 0} = α \frac{dC}{dt} \vert}_{t = 0} \qquad ( 2 )$$

Based on equation 2, the CO₂ concentration in cell culture medium is linearly proportional to the initial diffusion rate of the CO₂ through the silicone layer.

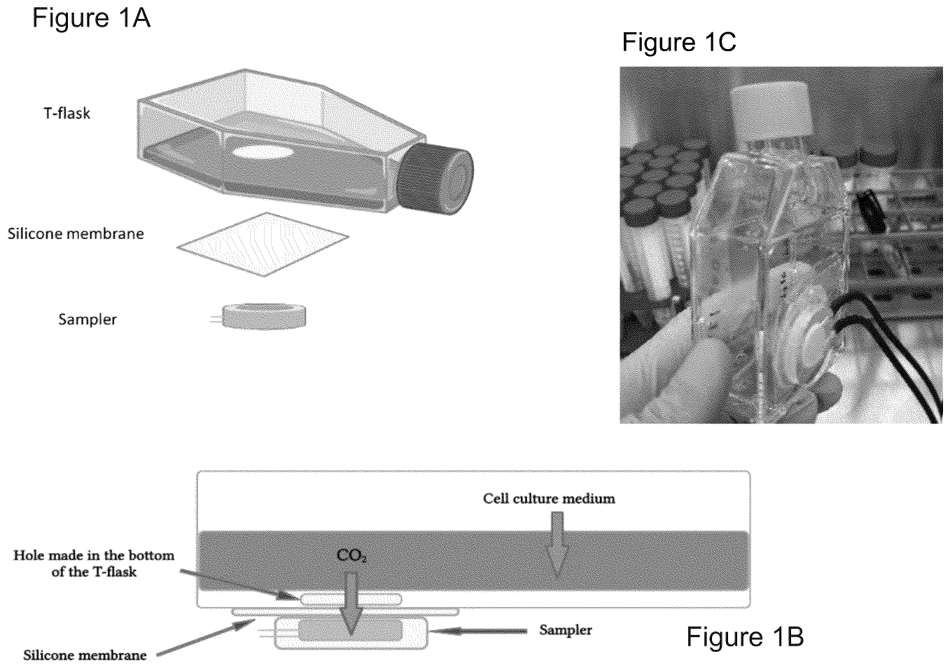

The present invention provides a new method to measure DCO₂ in a noninvasive way as shown in FIGS. 1A-1C. The idea was achieved by modifying a T-flask. In this method a small area of the vessel was replaced with silicone membrane by creating a hole in the bottom wall of the T-flask and attaching silicone membrane on the hole from outside of the T-flask. Silicone membrane is permeable to CO₂ and facilitates the diffusion of CO₂ during the cell culture process. A sampler is then attached on the silicone membrane from outside. The sampler has a cavity in its center. When attaching the sampler on the silicone membrane, the cavity is aligned with the hole created in the bottom wall of the T-flask. FIG. 1A shows the proposed design. During the cell culture process, when cell culture medium is present on the silicone layer, the CO₂ dissolved in the culture medium diffuses through silicone membrane. It is then collected in the sampler and circulated to the sensor for the measurements. [19]. These steps are shown in FIG. 1B. A modified T-flask equipped with noninvasive monitoring system for dissolved CO₂ is presented in FIG. 1C.

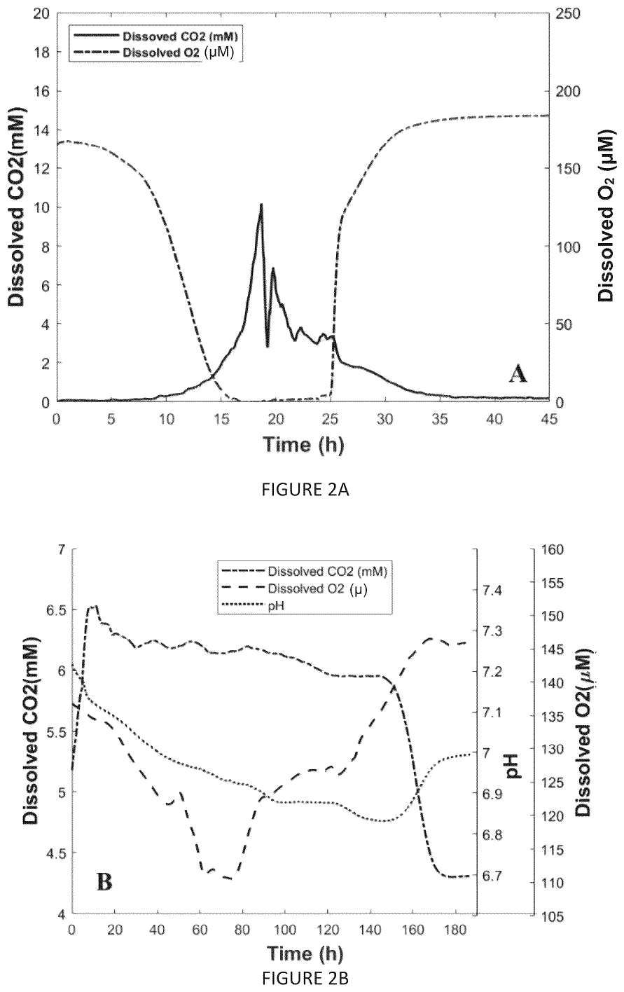

In the proposed design, the mass transfer happens through a silicone membrane attached on the hole created in the bottom wall of the T-flask. After completing the fabrication step, sterilization of T-flask was achieved by a microwaving method based on a previous study on reusing tissue culture vessels [22]. In this method, the T-flask was rinsed thoroughly with deionized (DI) water. The T-flask was then placed in a 2.45 GHz home type microwave and microwaved for 3 minutes. A container including 200 mL DI water was placed next to the T-flask during the microwave process to act as heat sink. 10 mL of LB broth medium was added to the modified and microwaved T-flask to evaluate the sterilization method. The T-flask was then placed in incubator set at 37° C. and 5% CO₂ and monitored for any sign of contamination over 7 days. No contamination was observed during this time. The method has been proven to be successful repeatedly in cell culture processes conducted in the modified T-flask. The noninvasive measurement of CO₂ was evaluated by culturing Pichia Pastoris in a modified T175-flask as proof-of-concept study. The design was also evaluated by culturing CHO cells in a modified T175 flask. The DO and the DCO₂ profiles obtained throughout the culture are presented in FIGS. 2A and 2B.

### Method for Noninvasive Measurement of pH

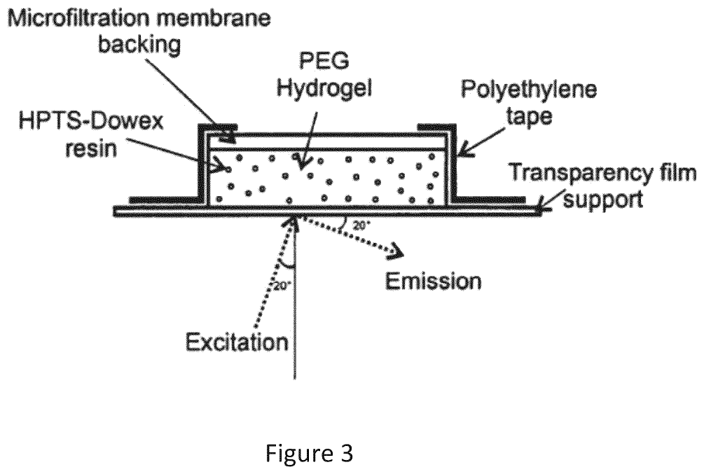

For measurement of pH, an optical sensor comprises a sensing patch and electronics. The fluorescent dye, 8-hydroxy-1,3,6-pyrene trisulfonic acid, is immobilized onto Dowex anion exchange resin. The resin is then entrapped into a hydrogel highly permeable to proton. This sensing layer is polymerized on a microfiltration membrane that provides a barrier to the fluorescence. FIG. 3 shows different parts of a pH patch.

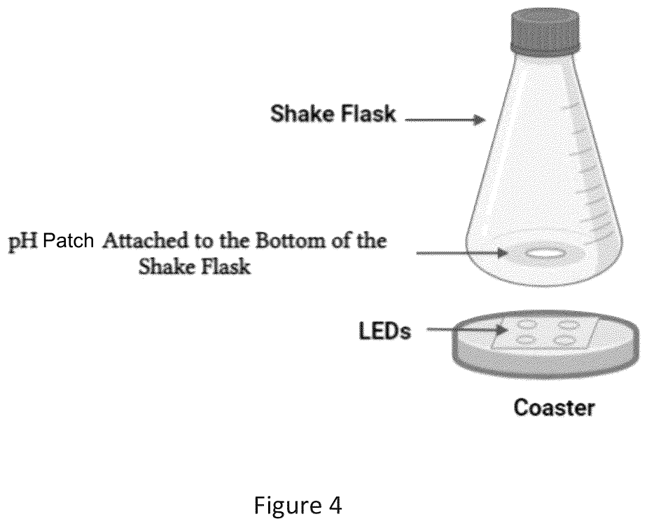

The absorbance spectrum of dye changes with respect to the pH, and the fluorescent indicator exhibits a shift in excitation or emission by change in the pH. The pH measurement via patches is ratio-metric detection method where the ratio of emission intensity at two excited wavelengths (468 nm and 408 nm) is defined as corrected ratio and correlated with the pH value of the buffer. FIG. 4 shows a noninvasive way by attaching a cellulose membrane and the patch attached on the outside of a shake flask and placed on the coaster for pH measurements [21]. Coaster is the electronic part of the sensor and the LED light on the coaster acts as an excitation source illuminating the patch. The fluorescent dye inside the patch is excited and emits light which will then be detected by detector and converted to readings displayed in software [20].

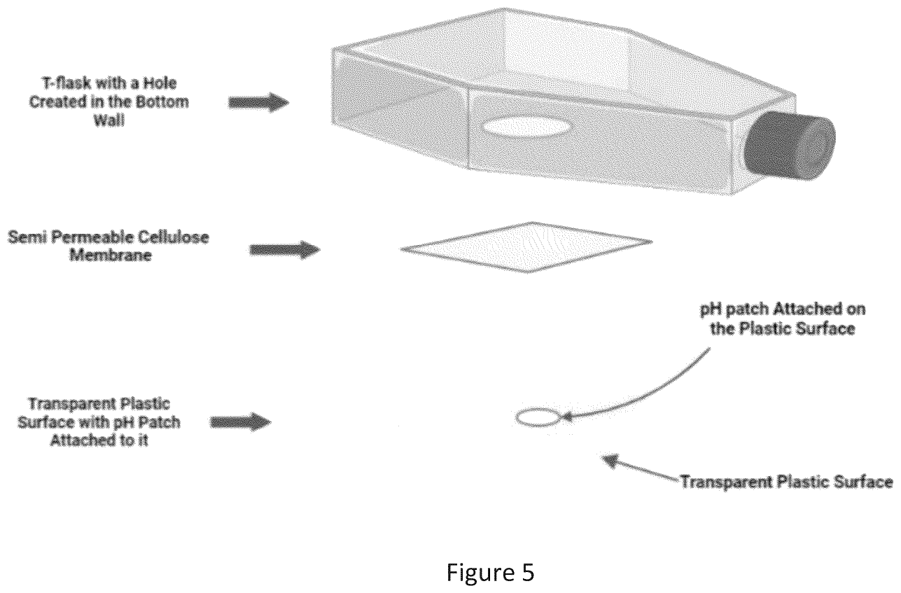

As stated above, the noninvasive measurement was achieved by placing the cellulose membrane between the pH patch and cell culture medium. The cellulose membrane is a semi-permeable membrane where all sample components move towards equilibrium concentration on both sides of the membrane. The membrane has a distinct molecular weight cut off (MWCO) of 12000 Daltons, and the pore size of 4.8 nm. In addition, it is stable within the pH range of 5-9. The new design was achieved by creating a hole in the bottom wall of a T-flask and attaching a cellulose membrane on the hole from outside of the T-flask. A pH patch was then attached on a transparent surface, and 200 μL of DI water was added on top of the patch. The modified T-flask was then placed on the transparent surface, with wet pH patch attached to it. This step was conducted carefully to align the patch with the cellulose membrane covering the hole. FIG. 5 shows the proposed design for noninvasive measurement of pH using a T-flask.

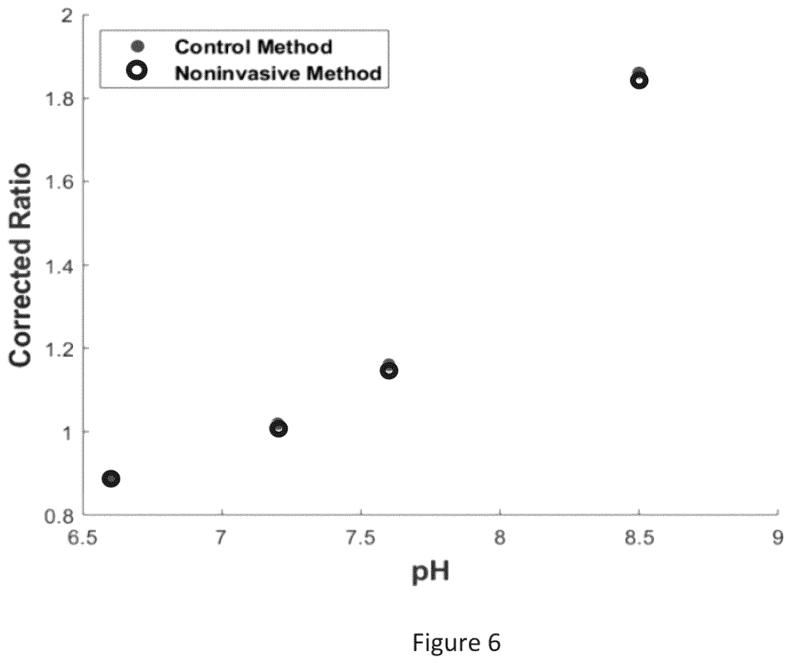

The noninvasive method for pH measurement was tested by adding solutions with different pH values to the modified T-flask. After reaching equilibrium, the corrected ratio was recorded for each solution. The T-flask was then removed, and the solutions were directly added to the pH patch. The corrected ratios were recorded for the condition where patch was in direct contact with the solutions. The results from two experiments are compared in FIG. 6.

As it can be seen in FIG. 6, the corrected ratios obtained via cellulose membrane are comparable with the corrected ratios obtained from direct contact of the pH patch to the solutions. Therefore, it can be concluded that the noninvasive pH measurement via cellulose membrane is effective. Furthermore, the response time for each method was measured. This experiment was conducted in two steps:
1—Measuring the response times for both methods when the solutions were added with decreasing order (8.5, 7.6, 7.2, and 6.6).
2—Measuring the response times when adding solutions with pH values in increasing order (6.6, 7.2, 7.6, and 8.5). The results are reported in Tables 1 and 2 respectively.

TABLE 1

| | Response Time (minutes) for: | | | |
|---|---|---|---|---|
| | pH = 8.5 | pH = 7.6 | pH = 7.2 | pH = 6.6 |
| Control | 40 | 13 | 11 | 10 |
| Via Cellulose Membrane | 50 | 20 | 13 | 15 |

Response times when solutions added from highest to lowest pH value for: 1: Control method (Adding the solution directly on the patch). 2: Noninvasive method (Adding the solution on the cellulose membrane).

TABLE 2

| | Response Time (minutes) for: | | | |
|---|---|---|---|---|
| | pH = 6.6 | pH = 7.2 | pH = 7.6 | pH = 8.5 |
| Control | 4 | 12 | 13 | 35 |
| Via Cellulose Membrane | 17 | 36 | 30 | 63 |

Response times when solutions added from lowest to highest pH value for: 1: Control method (Adding the solution directly on the patch). 2: Noninvasive method (Adding the solution on the cellulose membrane).

A comparison between the results from Tables 1 and 2 shows that the response time difference between the control method and noninvasive method is more noticeable under condition of adding pH solutions in increasing order in comparison to the case that the pH solutions were added in decreasing order. Despite this fact, the response times are acceptable for cell therapy purposes where cell growth rate is slow.

In the next study, the effect of long-term exposure of the cellulose membrane to the cell culture medium was investigate. This study was mainly conducted to address the concern over the adverse effect of cell culture medium on the integrity of the cellulose membrane, and consequently the sensor measurements. For this purpose, 10 mL of complete medium (90% v/v DMEM+10% v/v FBS) was added to the sterilized modified T-flask and placed in 5% incubator for 10 days. In day 10, the medium was removed from T-flask, and the T-flask was rinsed 3× using deionized water. Different solutions were then added to the T-flask and response times corresponding to each pH value were measured. Tables 3 and 4 show the response times for each measurement before and after 10 days of cellulose membrane contacting the cell culture medium.

TABLE 3

| | Response Time and Corrected Rations | | | |
|---|---|---|---|---|
| | Measured Before 10 days Contact with Medium | | | |
| | pH = 8.5 | pH = 7.6 | pH = 7.2 | pH = 6.6 |
| Corrected Ratio | 1.61 | 1.23 | 1.10 | 0.98 |
| Measured Before | | | | |
| 10 days Contact | | | | |
| with Medium | | | | |
| Response Time | 50 | 20 | 15 | 23 |
| Before 10 days | | | | |
| Contact with | | | | |
| Medium (Minutes) | | | | |

The response times and corrected ratios measured via noninvasive method (via cellulose membrane) before 10 days exposure of the cellulose membrane to the cell culture medium.

TABLE 4

| | Response Time and Corrected Rations | | | |
|---|---|---|---|---|
| | Measured After 10 days Contact with Medium | | | |
| | pH = 8.5 | pH = 7.6 | pH = 7.2 | pH = 6.6 |
| Corrected Ratio | 1.60 | 1.16 | 1.03 | 0.93 |
| Measured Before | | | | |
| 10 days Contact | | | | |
| with Medium | | | | |
| Response Time | 58 | 20 | 13 | 15 |
| After 10 days | | | | |
| Contact with | | | | |
| Medium (Minutes) | | | | |

The response times and corrected ratios measured via noninvasive method (via cellulose membrane) after 10 days exposure of the cellulose membrane to the cell culture medium

As it is shown in Tables 3 and 4, the response times and corrected ratios before and after 10 days exposure of the cellulose membrane to the cell culture medium are not significantly different. In other words, the proposed pH measurement via cellulose membrane is feasible and effective for long term cell culture processes.

In this study, A modified nonsterile modified T-flask was used for measurement of corrected ratios and response times. However, an identical modified T-flask was sterilized to study the long term effect of the cellulose contact with cell culture medium. The T-flask was sterilized by the microwave method described in previous section. In day 10, the T-flask was investigated, and no sign of contamination was observed. Therefore, it can be concluded that the sterilization method was successful in sterilizing the modified T-flask. Additionally, the results from sensor measurements (corrected ratios as well as the response times) indicate that the sterilization method does not affect the measurements.

A study was conducted to evaluate the continuous pH measurement via cellulose membrane. In this experiment, a pH patch was attached to the bottom wall of the modified T-flask from inside. A second patch from a different batch was attached to a transparent layer, and 500 μL of DI water was added on top of the patch. The wet patch was then placed under the cellulose membrane covering the hole in the bottom wall of the modified T³ flask. 20 mL of complete medium (90% v/v DMEM+10% v/v FBS) was added to the modified T-25 flask. The DMEM medium is buffered with CO₂/HCO-based buffer. This bicarbonate buffering system works based on the Le Chatelier's principle shown in equation below:

$${HCO}_{3}^{-} + {H}^{+} ↔ {CO}_{2} + {H}_{2} O \qquad ( 3 )$$

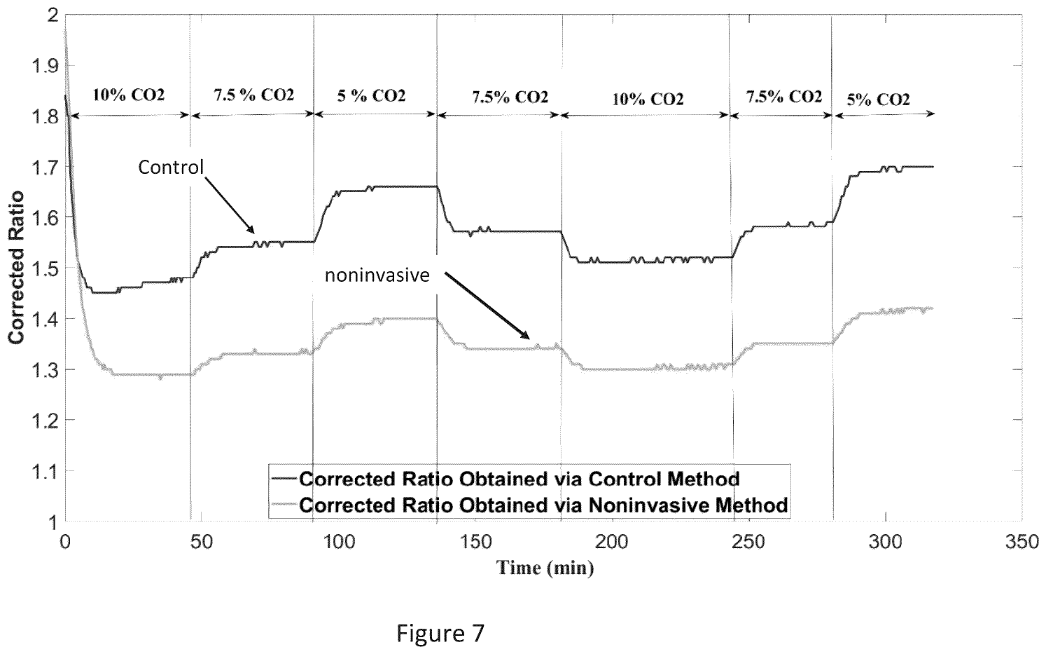

Based on the equation 3, an increase in the partial pressure from the CO₂ will result in an increase in the Concentration of H⁺, and consequently a lower pH. Therefore, sparging different percentages of CO₂ in the cell culture medium would result in a change in the pH level of the cell culture medium. This change, however limited, will be reflected in the corrected ratio calculated by the pH sensor. In other words, by sparging higher percentage of CO₂, pH value would decrease, and this could be observed in the decrease in the corrected ratio value. Similarly, sparging lower percentages of CO₂ would result in higher values of corrected ratio. The CO₂ sparging method was used to create a continuous change in pH value. The corrected ratios were measured via direct contact of patch with the cell culture medium and via cellulose membrane simultaneously. FIG. 7 shows the corrected ratio profiles for both methods.

Similar corrected ratio profiles are observed for both control and noninvasive method. Additionally, the delay observed for noninvasive measurement method is negligible. This indicates that the patch outside the cell culture medium was able to track changes happening in the cell culture medium.

### Method for Noninvasive Measurement of Dissolved Oxygen

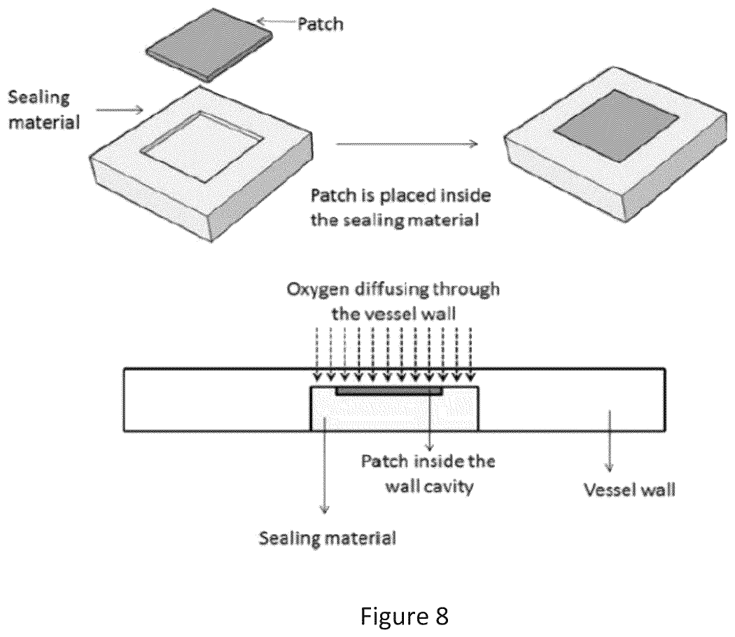

Dissolved oxygen (DO) sensor is measured using an optical sensor including electronics and the sensing patch. The patch consists of four layers. First layer is an acrylic copolymer and is used as an optical isolator. Below this layer, the sensing layer, has the fluorescent dye Tris (4,7-diphenyl-1,10 phenanthroline) ruthenium (II) dichloride immobilized in a silicone matrix. There is a support polyester layer below the second layer. Below the support layer, there is an adhesive layer that is used to attach the patch to the outside of the vessel. This layer is supported by a polyester layer below it. The optical DO sensor was used to monitor the dissolved oxygen from cell culture in a noninvasive way. In this method, the patch was placed inside a transparent sealing material placed outside the oxygen permeable wall of the vessel, the diffused oxygen was then detected by the sensor. FIG. 8 is a schematic demonstrating the patch placement outside the vessel.

The oxygen dissolved in the cell culture medium diffuses through the wall of the cell culture vessel and is detected by the patch inside the sealing. The idea was evaluated in cell cultures inside T-flask as well as a culture bag. The results show that the noninvasive method was successful in tracking the changes happening inside the cell culture medium, and the method is more appropriate for slow growing cell lines such as mammalian cells [8].

### Setup for Noninvasive Measurement of DO, pH, and DCO₂ Simultaneously

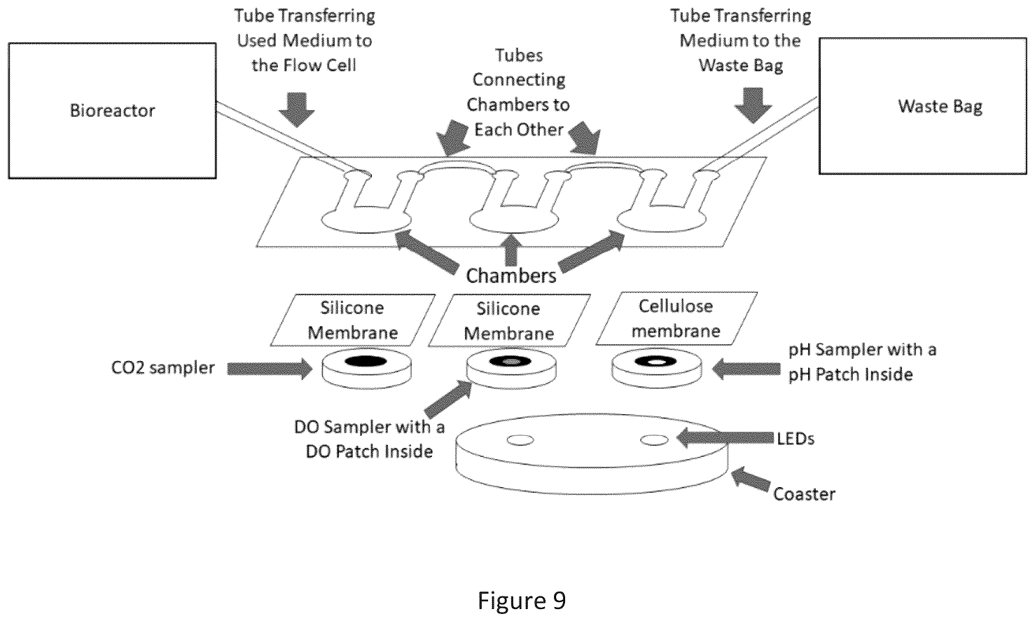

Different approaches to measure DO, DCO₂, and pH in a noninvasive way were described separately in previous sections. Hereinbelow, a design that combines all three ideas in one system is discussed. The proposed flow cell consists of three chambers. Each chamber has one inlet and one outlet and is connected to a membrane specific for measuring DO, DCO₂, or pH. In other words, the first chamber is covered with cellulose membrane, and is allocated for measuring pH. The second chamber is covered with silicone membrane. Silicone membrane is highly permeable to oxygen; therefore, the second chamber will be used for measuring DO. The third chamber is similarly covered with silicone membrane via which DCO₂ will be measured. Considering the order, for first chamber, a pH patch is attached to transparent surface and then attached to the other side of the cellulose membrane. For the second chamber, a DO patch is attached inside the cavity of a transparent sampler, the sampler is then attached to the other side of the chamber. Similarly, a sampler is attached on the other side of third chamber in a way that its cavity is aligned with the silicone membrane. The flow cell can be integrated with the any type of perfusion bioreactor such that the flow transferring the used medium from bioreactor to the waste is redirected to the flow cell, circulated in chambers. At this point the measurements are conducted for pH, DO and DCO₂. The used medium is then transferred from flow cell to the waste. The three chambers in the flow cell are interconnected in a way that the outlet of the first chamber is connected to the inlet of the second chamber. Similarly, the outlet of the second chamber is connected to the inlet of the third chamber. In order to integrate the flow cell with the bioreactor, the inlet of the first chamber is connected to the tube transferring the medium from bioreactor, and the third chamber is connected to the tube transferring the used medium to the waste. FIG. 9 shows a schematic of the setup.

### Noninvasive Measurement of DO, DCO₂, and pH

The flow cell is validated by conducting noninvasive measurement of DO, DCO₂, and pH via flow cell and comparing the results with the measurements obtained directly from inside the bioreactor. To conduct the validation study, an environment with changing levels of DO, DCO₂, and pH is created. Different levels of DO can be obtained by mixing pure N₂ and O₂ through two mass flow controllers, and sparging the gas mixture in the cell culture medium in the bioreactor. Similar method is used to create different percentage of CO₂. However, for this purpose CO₂ gas is used instead of O₂ gas. To create a dynamic change in pH value, different percentages of CO₂ sparged in the cell culture medium. The change of the level of CO₂ present in the cell culture medium results in change of pH value.

During the measurement process, the used medium is transferred to the flow cell. Then a specific volume of medium is collected in each chamber. A membrane is attached to the bottom of each chamber. Thus, CO₂, O₂ or protons will diffuse through their corresponding membranes. After this step, the measurements are conducted via DO, DCO₂, and pH sensors. COMSOL Multiphysics will be used to simulate the diffusion processes in all chambers and studying parameters such as the response time for the sensors. During this step, different parameters such as volume of sample required to conduct the measurements is optimized

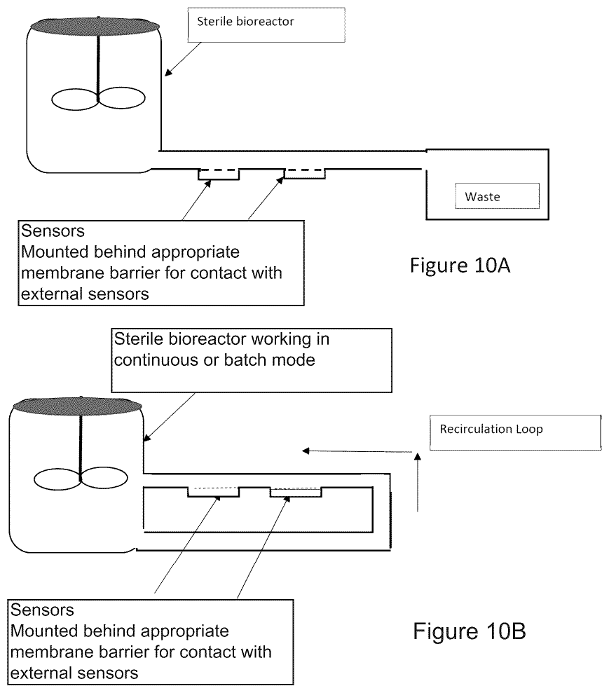

Further the present invention details a method and system using a recirculating loop connected to a bioreactor with appropriate barrier membrane ports that will enable completely sterile measurement of multiple parameters. FIGS. 10A and 10B demonstrate a simplified system using a waste line or recirculating loop. The main sampling tube/manifold is made of steam or radiation sterilizable material such as polycarbonate, polypropylene, stainless steel etc. Ports are present in the sampling manifold to allow for suitable barrier membranes to be placed that allow for particular species to be measured. For example, for sensing diffusible gas species such as oxygen, CO₂, ammonia etc., a silicone membrane barrier (it can be 1-10 microns thick and supported by a stainless steel or other mesh for strength) to allow for rapid diffusion. For pH, a nafion membrane (proton conducting) may be employed. For glucose and other small molecule analytes, a 1000 molecular weight cutoff (MWCO) dialysis membrane can be used. For peptides/antibodies, this could be a 1000-200,000 MWCO membrane. For ions or other smaller diffusible species, it could be cation or anion exchange membranes of <1000 MWCO. The cutoff is not to exceed a size that will allow ingress of microbes and viruses such the sampling manifold can be exposed to non-sterile conditions. Membranes can also be specified to minimize any leachables and extractable components from the sensor back-diffusing into the process. Notably the present invention provides the advantage that the sampling sensors need not be sterile because they are positioned outside of the bioreactor and can be removed for calibration or replaced if they malfunction without compromising the sterility of the interior. Furthermore, they can readily be replaced or recalibrated without disturbing the process. Various types of instrumentation (electrochemical, optical, acoustic, or other analytical modes can be used to interrogate the sensors and/or analyze the diffusible species crossing the membrane. In other embodiments, the sampling membrane can be made to be active transport elements to not rely solely on passive diffusion. Furthermore, on the distal side measurements can be made either by direct sensor insertion or by using the distal side as an equilibration chamber as described in U.S. Pat. No. 9,538,944 and the measurement made in a separate chamber. The advantage here is that larger and bulky instruments such as an HPLC can be used to sample for proteins and DNA. In addition, by using a purge cycle, rate-based sampling as described in '944 can be employed. The scale of the system can range from microfluidic to milli- and liter size systems in instances of large bioreactors.

Use in Human Mesenchymal Stem Cell (hMSC) Culture Process

Importantly the present invention is applicable for monitoring and measuring components in a human mesenchymal stem cell (hMSC) culture process. Studies have shown that hypoxia enhances hMSCs performance. Collected data shows a general enhancement in growth, attachment, genomic stability, paracrine activity, and cell surface markers. However, despite the consistent results in these areas, the results for differentiation of hMSCs vary. In other words, the results of these studies show a variety of outcomes when discussing the differentiation potential of hMSCs under hypoxia condition [24]. Hypoxic is a term used when cells are exposed to 0.5% to 10%02 level in culture headspace. However, temperature, pressure and salinity in the cell culture medium and cause different levels of dissolved oxygen. Considering the fact that available differentiation assays are qualitative and oxygen level is an important factor in differentiation of stem cells, the use of the present invention in the monitoring of DO in the cell culture medium will help in providing a better understanding of the cell culture environment and making more reliable conclusions regarding the effect of oxygen.

The present invention provides:
1—An accurate and quantitative DO level for differentiation of hMSCs; and
2: Analyzing the DCO₂, and pH profiles in stem cell culture and correlating them to the differentiation state of hMSCs. The results provide a more accurate and reliable information from hMSC cultures and enhance the understanding of differentiation of stem cells.

### REFERENCES

The contents of all references cited herein are incorporated by reference herein for all purposes.
1. Eaker, S., Abraham, E., Allickson, J., Brieva, T. A., Baksh, D., Heathman, T. R., & Zhang, N. (2017). Bioreactors for cell therapies: current status and future advances. Cytotherapy, 19(1), 9-18.
2. Stephenson, M., & Grayson, W. (2018). Recent advances in bioreactors for cell-based therapies. F1000 Research, 7.
3. Zhang, H., Kent, D. E., Albanna, M., Lhu, L., Sun, X. S., Eaker, S., & Somara, S. (2021). Bioreactor Technology for Cell Therapy Manufacturing in Regenerative Medicine. Current Stem Cell Reports, 7(4), 212-218.
4. Holzberg, T. R., Watson, V., Brown, S., Andar, A., Ge, X., Kostov, Y., & Rao, G. (2018). Sensors for biomanufacturing process development: facilitating the shift from batch to continuous manufacturing. Current opinion in chemical engineering, 22, 115-127.
5. SERIES, D. C. (2021) Should CO₂ Be a Critical Process Parameter?
6. Al-Ani, A., Toms, D., Kondro, D., Thundathil, J., Yu, Y., & Ungrin, M. (2018). Oxygenation in cell culture: Critical parameters for reproducibility are routinely not reported. PLOS One, 13(10), e0204269.
7. Rahmatnejad, V. (2021). Noninvasive Sensor Applications in Cell Culture (Masters dissertation. University of Maryland, Baltimore County).
8. Gupta, P. A., Ge, X., Kostov, Y., & Rao, G. (2014). A completely noninvasive method of dissolved oxygen monitoring in disposable small-scale cell culture vessels based on diffusion through permeable vessel walls. Biotechnology Progress, 30(1), 172-177.
9. Eaker, S., Armant, M., Brandwein, H., Burger, S., Campbell, A., Carpenito, C., & Wagey, R. (2013). Concise review: guidance in developing commercializable autologous/patient-specific cell therapy manufacturing. Stem cells translational medicine, 2(11), 871-883.
10. Jagschies, G., Lindskog, E., Lacki, K., & Galliher, P. M. (Eds.). (2018). Biopharmaceutical processing: development, design, and implementation of manufacturing processes. Elsevier.
11. Schmelzer, A. E., & Miller, W. M. (2002). Hyperosmotic stress and elevated pCO₂ alter monoclonal antibody charge distribution and monosaccharide content. Biotechnology progress, 18(2), 346-353.
12. Borys, M. C., Linzer, D. I., & Papoutsakis, E. T. (1993). Culture pH affects expression rates and glycosylation of recombinant mouse placental lactogen proteins by Chinese hamster ovary (CHO) cells. Bio/technology, 11(6), 720-724.
13. Monfoulet, L. E., Becquart, P., Marchat, D., Vandamme, K., Bourguignon, M., Pacard, E., . . . & Logeart-Avramoglou, D. (2014). The pH in the microenvironment of human mesenchymal stem cells is a critical factor for optimal osteogenesis in tissue-engineered constructs. Tissue Engineering Part A, 20(13-14), 1827-1840.
14. Tsai, C. C., Chen, Y. J., Yew, T. L., Chen, L. L., Wang, J. Y., Chiu, C. H., & Hung, S. C. (2011). Hypoxia inhibits senescence and maintains mesenchymal stem cell properties through down-regulation of E2A-p21 by HIF-TWIST. Blood, The Journal of the American Society of Hematology, 117(2), 459-469.
15. Jang, Y. Y., & Sharkis, S. J. A low level of reactive oxygen species selects for primitive hematopoietic. stem cells that may reside in the low-oxygenic niche. Blood. 2007 Oct. 15; 110(8):3056-63.
16. Miller, A. T. (1966). The role of oxygen in metabolic regulation. Helgoländer wissenschaftliche Meeresuntersuchungen, 14(1), 392-406.
17. Baker, M. (2016). 1,500 scientists lift the lid on reproducibility. Nature News, 533(7604), 452.
18. Schulz, E., & Münzel, T. (2011). Intracellular pH: a fundamental modulator of vascular function. Circulation, 124(17), 1806-1807.

2019. Chopda. V. R., Holzberg, T., Ge, X., Folio, B., Tolosa, M., Kostov, Y., & Rao, G. (2020). Real-time dissolved carbon dioxide monitoring I: Application of a novel in situ sensor for CO₂ monitoring and control. Biotechnology and Bioengineering. 117(4), 981-991.
20. Vallejos, J. R., Brorson, K. A., Moreira, A. R., & Rao, G. (2010). Dissolved oxygen and pH profile evolution after cryovial thaw and repeated cell passaging in a T-75 flask. Biotechnology and bioengineering, 105(6), 1040-1047. 25
21. Hanson, M. A., Ge, X., Kostov, Y., Brorson, K. A., Moreira, A. R., & Rao, G. (2007). Comparisons of optical pH and dissolved oxygen sensors with traditional electrochemical probes during mammalian cell culture. Biotechnology and bioengineering, 97(4), 833-841.
22. Sanborn, M. R., Wan, S. K., & Bulard, R. (1982). Microwave sterilization of plastic tissue culture vessels for reuse. Applied and environmental microbiology, 44(4), 960-964.
23. Wiegmann, V., Martinez, C. B., & Baganz, F. (2020). Using a parallel micro-cultivation system (micro-matrix) as a process development tool for cell culture applications. In Animal Cell Biotechnology (pp. 69-81). Humana, New York, NY.
24. Bahsoun, S., Coopman, K., Forsyth, N. R., & Akam, E. C. (2018). The role of dissolved oxygen levels on human mesenchymal stem cell culture success, regulatory compliance. and therapeutic potential. Stem Cells and Development. 27(19). 1303-1321.
25. Weigi. Bernard H. et al . . . (1994) Optical triple sensor for measuring pH. oxygen and carbon dioxide. Journal of Biotechnology. V 32. pp 127-138.

That which is claimed is:

1. A noninvasive system for monitoring and/or measuring testing parameters within a bioprocess medium, the noninvasive system comprising:
a bioprocess container for holding the bioprocess medium, wherein the bioprocess container comprises an (i) opening in a wall of the bioprocess container for monitoring and/or measuring testing parameters diffusing from the bioprocess container, (ii) a waste line attached to the bioprocess container for movement of fluid comprising the testing parameters from the bioprocess container or (iii) a recirculation loop for movement of fluid comprising the testing parameters to and from the bioprocess container;
a semi-permeable membrane communicatively connected to a sampler receptor wherein the semipermeable membrane is positioned between the waste line, opening or recirculation loop and the sampler receptor and allows for movement of testing parameters into the sampler receptor; and
a sensor communicatively connected to the sampler receptor for measuring and/or monitoring the testing parameters within the bioprocess medium.

2. The noninvasive system according to claim 1, wherein the testing parameters are selected from the group consisting of dissolved carbon dioxide (DCO₂), dissolved oxygen (DO) and pH.

3. The noninvasive system according to claim 1, wherein all sensors are positioned outside of the bioprocess container and testing is conducted outside of the bioprocess container.

4. The noninvasive system according to claim 1, wherein the bioprocess container and lines extending therefrom are fabricated with nonpermeable materials.

5. The noninvasive system according to claim 1, wherein the waste line, opening or recirculation loop include flow controls to control the flow of fluids from the bioprocess container.

6. The noninvasive system according to claim 1, wherein the semipermeable membrane for testing of DCO₂ and DO is fabricated of silicone and testing for pH comprises cellulose.

7. The noninvasive system according to claim 1, wherein the sensor is a sensor for pH and DO and DCO₂ components.

8. The noninvasive system according to claim 7, wherein the pH sensor is a pH patch communicatively connected to a cellulose membrane, wherein the pH patch comprises a fluorescent dye.

9. The noninvasive system according to claim 7, wherein the sensor for measuring the DO and DCO₂ components is an optical sensor including electronics and a sensing patch comprising a fluorescent dye immobilized in a silicone matrix.

10. A noninvasive system for monitoring and/or measuring dissolved O₂ (DO), pH and dissolved CO₂ (DCO₂) within a bioprocess medium, the noninvasive system comprising:
a bioprocess container for holding the bioprocess medium, wherein the bioprocess container is connected to a waste line or recirculation loop line for movement of fluid from the bioprocess container;
at least one sampling chamber for each testing parameter, wherein the sampling chamber has an inlet and outlet communicatively connected to the waste line or recirculation loop, wherein the outlet is communicatively connected to at least one sampler receptor;
a semi-permeable membrane positioned between the outlet of the sampling chamber and the sampler receptor and allows for movement of fluid from the sampling chamber into the at least one sampler receptor; and
a sensor communicatively connected to the at least one sampler receptor for measuring and/or monitoring the testing parameters of at least dissolved O₂, pH and dissolved CO₂ within the bioprocess medium.

11. The noninvasive system according to claim 10, wherein sampler receptors collect the medium for testing of the components of DO, DCO₂, and pH, after the bioprocess medium passes through the semi-permeable membranes.

12. The noninvasive system according to claim 10, wherein the non-invasive system comprises three sampling chambers each having one inlet and one outlet, wherein the outlet is connected to a semipermeable membrane specific for measuring the components of DO, DCO₂, and pH.

13. The noninvasive system according to claim 12, wherein the outlet of one sampling chamber is covered with a cellulose membrane for measuring pH, wherein the outlet of another sampling chamber is covered with a silicone membrane for measuring dissolved oxygen and the outlet of a third sampling chamber is covered with a silicone membrane for measuring DCO₂.

14. The noninvasive system according to claim 10, wherein all testing is conducted outside of the bioprocess container and all sensors are positioned outside of the bioprocess container.

15. The noninvasive system according to claim 10, wherein the bioprocess container, sampling chambers and waste line or recirculating loop are fabricated from nonpermeable materials.

16. The noninvasive system according to claim 10, wherein the waste line or recirculation loop include flow controls to control the flow of fluids from the bioprocess container into the waste line or recirculation loop.

17. The noninvasive system according to claim 10, wherein three sensors are used for testing, a pH sensor for pH testing, a DO sensor for sensing DO and a DCO₂ sensor for sensing DCO₂.

18. The noninvasive system according to claim 17, wherein the pH sensor is a pH patch communicatively connected to a cellulose membrane, wherein the pH patch comprises a fluorescent dye.

19. The noninvasive system according to claim 17, wherein the sensor for measuring the DO and DCO₂ components is an optical sensor including electronics and a sensing patch comprising a fluorescent dye immobilized in a silicone matrix.

20. A method for noninvasive monitoring and/or measuring testing parameters selected from dissolved carbon dioxide (DCO₂), dissolved oxygen (DO) and pH within a bioprocess medium, the method comprising:
i) providing a noninvasive testing system comprising:
a) a bioprocess container for holding the bioprocess medium, wherein the bioprocess container is connected to a waste line and/or recirculation loop line for movement of fluid from the bioprocess container;
b) at least one sampling chamber for each testing parameter, wherein the sampling chamber has an inlet and outlet communicatively connected to the waste line and/or recirculation loop, wherein the outlet is communicatively connected to at least one sampler receptor;
c) a semi-permeable membrane positioned between the outlet of the sampling chamber and the sampler receptor and allows for movement of fluid from the sampling chamber into the at least one sampler receptor; and
d) a sensor communicatively connected to the at least one sampler receptor for measuring and/or monitoring the testing parameters of at least dissolved O₂, pH and dissolved CO₂ within the bioprocess medium;
ii) moving/flowing the bioprocess medium from the bioprocess container into the at least one sampling chamber, wherein the bioprocess medium passes through the outlet of the sampling chamber and through the semi-permeable membrane, wherein the semipermeable membrane is fabricated of silicone or cellulose that is permeable to DCO₂, DO and pH;
iii) measuring the testing parameter in the sampler receptor with testing sensors.
